(19) United States
(12) Patent Application Publication
(10) Pub. No.: US 2004/0121025 A1
(43) Pub. Date: Jun. 24, 2004

(54) Oral composition for the treatment and prevention of bone loss

(76) Inventor: Dwight McKee, Aptos, CA (US)

Correspondence Address: WILLIAM J. SAPONE, COLEMAN SUDOL SAPONE P.C., 714 COLORADO AVENUE, BRIDGE PORT, CT, 06605, US

(21) Appl. No.: 10/325,683

(22) Filed: Dec. 20, 2002

(51) Int. Cl. A61K 35/78; A61K 33/06; A61K 33/26; A61K 31/714; A61K 31/7076

(52) U.S. Cl. 424/646; 424/682; 424/757; 514/52; 514/350; 514/89; 514/46; 514/251

(57) ABSTRACT

The invention is a composition and method for treating osteoporosis, bone loss and/or periodontal demineralization involving the oral administration of a formulation containing vanadium and ipriflavone which support an increase in bone mass, a homocysteine inhibitor/reducer to inhibit the counteracting effects of homocysteine on bone rebuilding, and an absorbable calcium salt to provide a ready source of material for bone rebuilding.

### CROSS REFERENCE TO RELATED APPLICATION

This application claims priority in U.S. Provisional Patent Application No. 60/342,808 filed Dec. 20, 2001.

### TECHNICAL FIELD

This invention relates to a composition and method for supporting the rebuilding of lost bone mass and for promoting and maintaining healthy bone mass.

### BACKGROUND

Bone loss can occur for various reasons. Simple aging can result in a thinning of bone mass over time, while various disorders such a osteoporosis result from accelerated bone loss. Various treatments, such as the use of corticosteroid therapy for asthma and autoimmune disease can result in a rapid loss of bone mass.

Among the compositions proposed as being useful for treating bone loss is ipriflavone (7-isopropoxysioflavone), a flavanoid found naturally in small quantities in various foods.

In U.S. Pat. No. 5,504,104, oral pharmaceuticals comprising oily vehicles for delivering ipriflavone are discussed for treating osteoporosis, the composition administered in capsules.

In U.S. Pat. No. 5,571,186, ipriflavone is formulated as a paste with hydroxylapatite or tricalcium phosphate, for use in filling bone defects.

The search continues for formulations which can enhance or supplement the bioavailability of ipriflavone, improve effectiveness in the oral administration of ipriflavone and generally provide a safe and effective formulation for supporting healthy bone mass.

### SUMMARY OF THE INVENTION

It is an object of the present invention to provide an oral formulation for promoting bone building which is effective in delivering ipriflavone together with complementary ingredients to safely and effectively maintain healthy bone mass.

It is another object of the present invention to provide an oral formulation for promoting bone building which is effective in substantially limiting the effects of aging or diseases on bone mass, such as osteoporosis or as a side effect to drug therapy.

These and other objects of the present invention are achieved by an oral formulation comprising ipriflavone together with a vanadium salt, a homocysteine inhibiting agent and an absorbable calcium salt.

Bone loss prevention, and rebuilding are accomplished by using ipriflaveone together with a vanadium salt. The two agents are believed to provide a synergistic effect. The homocysteine inhibitor is believed to block the detrimental effects of homocysteine on bone tissue, to increase the effectiveness of the bone loss prevention/bone rebuilding promoting components. The absorbable calcium provides a readily available source of material for replacing/rebuilding bone tissue, again to increase the effectiveness of the ipriflavone and vanadium salt combination.

Preferably, the formulation contains form 50 to 99% by weight ipriflavone, from 0.01-0.2% vanadium salt, from 0.1 to 2.0% homocysteine inhibitor and from 25-66% calcium salt.

The preferred vanadium salt is vanadyl sulphate, though others such as vanadium pentoxide, can be used. The homocystein inhibitor is preferably one or more folic acid, vitamin B12, vitamin B6, and methyl donors including, but not limited to, betaine (trimethylglycine), choline (tetramethylglycine), dimethylglycine, S-adenosyl-methionine (SAMe), S-adenosyl-cobaalamine (dibencozide), and methyl sulfonyl methan (MSM). The calcium salt is preferably calcium citrate/malate/carbonate, though other calcium salts may be used.

The method according to the present invention, provides for administering the inventive formulation for counteracting the effects of aging or osteoporosis on bone loss, in an effective manner with no significant side effects from long term use.

### DETAILED DESCRIPTION OF THE INVENTION

The invention relates to a formulation for the treatment of symptoms associated with, for example, osteoporosis, hypocalcemia and periodontal demineralization, using vanadium salts and ipriflavone in the presence of a complementary homocysteine inhibitor and absorbable calcium. The term “homocysteine inhibitor” is intended to refer to compounds that limit the effects of homocysteine on bone mass, for example, by reducing or recycling homocysteine to methionine and/or cysteine, though other mechanisms may also be effective. As the primary ingredients, vanadium salts promote the healthy formation of bone, and together with ipriflavone, promote the incorporation of calcium into bone while also inhibiting bone breakdown. The homocysteine inhibitor prevents homocysteine from counteracting the beneficial effects of the primary ingredients.

Clinical studies have shown that ipriflavone with supplemental calcium is effective in halting bone demineralization which leads to osteoporosis in postmenopausal women. Osteoporosis is a disease of bones characterized by loss of bone minerals (calcium, magnesium, trace minerals) and bone matrix (collagen, elastin, and other proteins), resulting in brittle bones which increases the risk of bone fracture from minimal trauma. According to the National Institute on Aging, 28 million middle-aged and older people are at risk of osteoporosis. In an older person with osteoporosis, fractures of the hip from falling is a leading cause for entry in nursing homes. Twenty percent of elderly people with osteoporosis who fracture a hip die within a year from complications set in motion by the fracture.

Ipriflavone promotes the incorporation of calcium into bone and also inhibits bone breakdown. Treatment with ipriflavone and supplemental calcium has been shown to be both safe and effective in halting bone loss in post-menopausal women or women who have had their ovaries removed as well as in improving bone density in cases of osteoporosis.

An animal study found ipriflavone inhibited a number of compounds known to be involved in increased resorption of bone. These include parathyroid hormone, vitamin D, prostaglandin E2, and interleukin 1-beta (Tsutsumi N, et al., Jpn J Pharmacol 1994;65:343-349). It was found that parathyroid-stimulated osteoclastic activity and resulting hypercalcemia were inhibited in a dosedependent manner by ipriflavone supplementation in rats (Bonucci E et al., CalcifTissue Int 1992;50:314-319).

Ipriflavone does not exhibit estrogen like effects, unlike many of the other isoflavones (Melis G B et al., J endocrin Invest 1992;15:755-761.). Ipriflavone has been shown to enhance the effects of estrogen on bone (Agnusdei D et al., Osteoporos Int 1995; 5:462466.), since ipriflavone appears to work by an entirely different mechanism than does estrogen.

Ipriflavone appears to be particularly effective for the treatment of osteoporosis in elderly women over the age of 65 (Agnusdei D, Bufalino L., Calcif Tissue Int 1997;61:S23-S27.; Passeri M et al., Bone Miner 1992;19:S57-S62.). Almost all of the clinical studies of ipriflavone in osteoporosis have been done in women, because clinically significant osteoporosis is much more common in women. Older men, particularly those on hormonal treatments for prostate cancer (Daniell H W, Urology August 2001;58 (2 Suppl 1):101-6.), also develop osteoporosis (Pande I, Francis R M, Baillieres Best Pract Res Clin Rheumatol July 2001; 15 (3):415-27.). Ipriflavone is also effective in preventing bone loss in men (Sato Y, Arch Phys Med Rehabil January 2000;81(1):117-21.).

The same protocol was used throughout most of the studies: 200 mg ipriflavone or placebo three times daily. In most of the studies, calcium (500-1000 mg) was given to both ipriflavone and placebo groups. Several two-year clinical trials studied women immediately post-menopause (age 50-65) and found bone mass was maintained or improved slightly in the ipriflavone groups while those in the placebo groups experienced significant bone loss (Gennari C et al., CalcifTissue Int 1997;61:SI9-S22; Agnusdei D et al., CalcifTissue Int 1997;61:142-147.; Valente M et al., Calcif Tissue Int 1994;54:377-380.).

Ipriflavone has been found to enhance the effect of other bone-preserving agents, including a form of vitamin D commonly used for osteoporosis (Ushiroyama T et al., Int J Gynaecol Obstet 1995;48:283-288). A number of studies have examined the combined effects of ipriflavone and estrogens for the treatment of osteoporosis. It has been found that lower (0.15-0.30 mg/day) doses of estrogen are equally effective in maintaining bone mass as are higher doses (0.625 mg/day or higher) when ipriflavone is included (Melis G B et al., Bone Miner 1992; 19:S49-S56.; Gambacciani M et al., Maturitas 1997;28:75-81.; Agnusdei D et al., Osteoporos Int 1995;5:462-466.).

Researchers have studied the effect of ipriflavone in avoiding bone loss induced by gonadotropin hormone-releasing hormone drugs such as Lupron®, which are widely used to induce reversible ovarian atrophy in premenopausal women for the treatment of endometriosis, uterine fibroids, or estrogen sensitive malignancies, such as breast carcinoma. In a study of women treated with Lupron for six months, a group was assigned to receive either ipriflavone or placebo in addition to supplemental calcium. In placebo subjects, bone mineral density decreased significantly by the end of the treatment. Conversely, there were no changes in bone density in the ipriflavone-treated group (Gambacciani M et al., Calcif Tissue Int. 1997;61:15-18.).

Ipriflavone has also been studied for its ability to prevent the rapid bone loss that follows surgical removal of the ovaries in premenopausal women. The results demonstrate that ipriflavone prevents the rapid bone loss that follows ovariectomy. Ipriflavone can be an attractive alternative for the prevention of osteoporosis in postmenopausal women who present contraindications to the estrogen replacement therapy (Gambacciani M et al., J Endocrinol Invest 1993; 16(5):333-337.).

Animal studies have found that ipriflavone inhibited bone loss associated with long term steroid use (Yamazaki I et al., Life Sci 1986:38:951-958.) and immobilization (Foldes I et al., Acta Morphologica Hungarica, 1988;36:79-93.; Notoya K et al., Calcif Tissue Int 1996;58:88-94.). Studies of ipriflavone in patients requiring long term corticosteroid therapy for asthma and autoimmune diseases would be of great interest, because steroid-induced osteoporosis is a major problem in this population.

Vanadium is the second primary ingredient in the inventive formulation. Vanadium is a trace mineral that is essential in humans but its exact role has not yet been defined. In many higher animals it is an essential nutrient, acting as a cofactor that enhances or inhibits the function of specific enzymes. In a study done with goats, female goats fed carefully formulated vanadium-deficient diets produced second-generation goat kids with skeletal damage that didn't survive beyond 3 days after birth (Harland B V, Harden-Williams B A, J Am Diet Assoc August 1994;94(8):891-4.; Amano R et al., J Trace Elem Med Biol September 1996; 10(3):145-8.). Like ipriflavone, vanadium has been shown to prevent bone resorption in laboratory experiments, and was equally effective against several different stimulators of bone resorption, including parathyroid hormone and prostaglandin E2 (Krieger N S, Tashjian A H Jr., Endocrinology July 1983; 113(1):324-8.).

The inventive formulation also includes a homocysteine inhibiting agent, preferably selected from one or more of the group consisting of Folic acid, vitamin B6, vitamin B 12 and methyl donors including, but not limited to, betaine (trimethylglycine), choline (tetramethylglycine), dimethylglycine, S-adenosyl-methionine (SAMe), S-adenosyl-cobalamine (dibencozide), and methyl sulfonyl methane (MSM). These are believed to function as cofactors for enzymes that can lower homocysteine levels. Homocysteine is a normal breakdown product of the amino acid methionine, which must be converted back into methionine, or to the amino acid cysteine. It is believed to exert a number of toxic effects in the body. A growing body of evidence suggests that an elevated homocysteine level is a risk factor for heart disease (Stampfer M J et al., JAMA 1992; 268:8778 I.; Bostorn A G et al., Arch Intern Med 1999; 159:1077-80). This association may represent a causeeffect relationship, as homocysteine appears to be capable of promoting the development of atherosclerosis. Increased homocysteine levels also appear to be a risk factor for the development of stroke (Perry I J et al., Lancet 1995; 346:1395-8). Elevated homocysteine levels is also believed to play a role in the development of osteoporosis (Brattstrom L E et al., Metabolism 1985; 34:1073-7). A number of studies have shown that supplementing with folic acid, vitamin B6, and vitamin B 12, (+/−methyl donor nutrients) can reduce homocysteine levels (Ubbink J B et al., Am J Clin Nutr 1993;57:47-53; Ubbink J B et al., J Nutr 1994; 124:1927-331; Dierkes Jet al., Int J Vitam Nutr Res 1998;68:98-103). While there has been some discussion that the reduction of postmenopausal risk of osteoporosis and cardiovascular disease can be accomplished through the use of ipriflavone and a comprehensive bone remineralization vitamin and mineral program which includes the use of folic acid, vitamin B6 and vitamin B 12 to control homocysteine levels (Kass-Annese B, Clin Obstet Gynecol March 2000;43(1): 162-83), a particular formulation to accomplish the goal had not been determined.

The inventive formulation also contains the raw material to rebuild bone mass. Calcium salts are included, preferably calcium citrate/malate/carbonate (CCMC), which appears to be the best absorbed of the many available forms of calcium (Miller J et al., Am J Clin Nutr 1988;48:12914.; Smith K T et al., Calcif Tiss Int 1987;41:351-2). When significant bone loss has already occurred, it is considered important to provide not only ipriflavone but the most bioavailable form of calcium to enhance the capability of the primary ingredients to increase bone mass. There is some evidence that CCM and CCMC maybe the most effective form of calcium in supporting maintenance of bone mass (Dawson Hughes B et al., N Engl J Med 1990;323:878-83.), and so they are the preferred calcium salts in the inventive formulation. Other salts would be calcium carbonate, citrate, fumarate, malate, succinate, aspartame, gluconate, caltrate and phosphate.

Taking the inventive formulation with an evening meal, or at bedtime with a snack, rather than in the early part of the day appears better for osteoporosis prevention or therapy than taking the formulation in the morning, based on the circadian rhythm of bone metabolism (Blumsohn A et al., J Clin Endocrinol 1994;79:730-5).

While there are several different medical/pharmaceutical options for the prevention and treatment of osteoporosis, these have various drawbacks. For example, hormone replacement therapy for women, which usually involves equine estrogens, and synthetic progestins, may be associated with increased risks of breast cancer (Vecchia C L et al., Maturitas Aug. 25, 2001;39(2):97115.). Others, such as Raloxifene (Evista®) a tamoxifen-like drug that has bone building effects and reduced risk of breast cancer may increase hot flashes; and the bisphosphonate medicines, such as acendronate (Fosmax®) etidornate (diaronel) and risendronate (actonel) build bone, but can produce irritating effects on the upper gastrointestinal tissues. Options for men with bone loss include bisphosphonate drugs and testosterone replacement if levels of the male hormone are low, but testosterone replacement may also be associated with an increase in the risk of prostate cancer (Slater S, Oliver R T, Drugs Aging December 2000; 17(6):431-9.). It is expected that the inventive formulation can supplant or support such pharmaceutical interventions, possibly enabling lower dosage treatment with reduced side effects, and it is believed the present formulation can be safely combined with these treatments.

The inventive formulation containing the ingredients discussed, ipriflavone, folic acid, vitamin B 12, vitamin B6, and methyl donors including, but not limited to, betaine (trimethylglycine), choline (tetramethylglycine), dimethylglycine, S-adenosyl-methionine (SAMe), S-adenosyl-cobalamine (dibencozide), and methyl sulfonyl methane (MSM), vanadium salt, and calcium salt, preferably calcium citrate/malate/carbonate, provide a nutritional option for maintaining and improving bone health that is without side effects or significant health risks, and which can also be used along with the pharmaceutical options. Lowering of homocysteine levels, in addition to improving bone health, also has the possibility of decreasing risk of other diseases of maturity.

The blend can be administered in various oral forms, which may include rapid release or sustained release or timed release formulations, such as tablets, lozenges, capsules pills, powders, granules, elixirs, tinctures, solutions, suspensions, syrups, and emulsions. The blend can include various diluents, excipients or carriers (hereafter referred to collectively as “carriers”), consistent with the desired route of administration.

The composition can include various optional ingredients that complement or support the bone building activity of the invention, or which support health in general. Among these optional ingredients are included other isoflavones such as from soy, red clover, or kudzu, pyridoxal-5phosphate, pyridoxine, various antioxidants, such as the oligomeric proanthocyanadins, vitamins, minerals, fiber, anti-inflammatory agents and other components provided these do not detrimentally affect the bone building promotion activity of the present invention or result in undesirable side effects. Other typical formulating ingredients such as binders, coloring or flavoring ingredients may also be used.

Generally, as an exemplary formula, a dose containing 300 mg ipriflavone, 50 mcg elemental vanadium (as vanadyl sulphate), 200 mcg folic acid and 350 mg calcium citrate/malate/carbonate administered twice per day is believed sufficient to promote bone rebuilding and/or otherwise maintain healthy bone mass.

The following are the relative ranges of ingredients usable in the invention:

ipriflavone 150 to 600 mg/day, more preferably 300 to 600 mg/day; vanadium salt, 50 to 1000 mg per day, more preferably 80 to 800 mcg/day; the homocysteine inhibiting agent(s), 1 to 20 mg per day, more preferably 5 to 20 mg/day; the absorbable calcium salt, 100-1000 mg, more preferably about 200-1000 mg/day.

The term “dose” as used here refers to physically discrete units suitable as unitary dosages for human patients and other warm blooded animals, each unit containing a predetermined quantity of the composition believed to produce the desired therapeutic effect in association with a physiologically tolerable carrier, e.g. a diluent or a vehicle. The specifications for the dosage forms of this invention are dictated by and are directly dependent on (a) the unique characteristics of the composition and the effect to be achieved, and (b) the limitations inherent in the art of compounding such a composition for use in humans and animals. Examples of suitable dosage forms in accord with this invention are tablets, capsules, pills, powder packets, granules, wafers and the like, segregated multiples of any of the foregoing, as well as solutions, emulsions and suspensions. Enteric coating is possible for oral ingestion.

The amount to be administered depends on the age and weight of the patient, the particular condition to be treated, the frequency of administration, and the route of administration. Veterinary dosages will correspond to human dosages with the amounts administered being in proportion to the weight of the animal as compared to adult humans.

A typical formulation may comprise the following:

### EXAMPLE 1

Bone Remineralization Formula:

Serving Size: 2 tablets. 2 servings a day.

1. Ipriflavone, 300 mg. (Range 300-600 mg. per day).

2. Vanadyl sulfate, 50 mcg. (Range 80-1000 mcg.).

3. Folic acid, 200 mcg. (50-1000 mcg.).

4. Vitamin B 12 (Cyanocobalamin) 100 mcg. (Range 25-1000 mcg.)

5. Pyridoxal-5-phosphate, 15 mg. (Range 5-100 mg.).

6. Calcium citrate/malate, (25%) 350 mg. (Total calcium 205 mg.) (range 100-1000 mg.).

7. excipients and formulating base.

While oral ingestion is the preferred route of administration, bone loss and remineralization are particular concerns in the mouth and the formula may be integrated into a toothpaste, mouth rinse or lozenge to promote periodontal health, by supporting remineralization of tooth surfaces through direct administration. The following formulas are examples of a toothpaste, mouth rinse and lozenge that may incorporate the inventive formulation, but the invention is not limited to these specific formulations and other combinations would fall within the scope of the present invention.

A gel dentifrice may have the following composition (typically, 1 gram is used with each application of a dentifrice to teeth and gums):

| | |
|---|---|
| Vehicle/humectant (e.g., glycerin, propylene | 40% |
| glycol, sorbitol) | |
| Abrasive (preferred is silica) | 15% |
| Binder/thickener (carbopol, CMC, HEC, xanthan gum) | 1% |
| Surfactant (sodium lauryl sulphate, pluronic F-127) | 1% |
| Optional ingredients including preservatives, flavoring | 1% |
| Agents, sweeteners, and coloring agents | |
| Ipriflavone | 15% |
| Vanadyl Sulphate | 0.005% |
| Folic Acid | 0.020% |
| Calcium Citrate/Malate | 20% |
| Deionized Water | q.s. to 100% |
| | by client |

An exemplary mouthrinse composition can be prepared according to conventional means containing the following ingredients:

| Surfactant (e.g., Pluronic F-127) | 1.75% | |
|---|---|---|
| Solubilizer (Ethyl Alcohol) | 6.00% | |
| Glycerin | 8.00% | |
| Preservatives, flavor, sweeteners, | 1.00% | |
| and coloring agents | | |
| Ipriflavone | 2.00% | |
| Vanadyl Sulphate | 0.0005% | |
| Folic Acid | 0.0020% | |
| Calcium Citrate/Malate | 2.00% | |
| Deionized Water | q.s. to 100.00% | |
| Dentifice | Mouthwash | |
| 1.5 mg | 2 | mg ID |
| .5 mcg | .5 | mcg UD |
| 2.0 mcg | 2.0 | mcg Folic |
| 20 mg | 2 | mg CA |

Application would proceed in customary fashion during brushing and mouth rinsing, and provides a convenient way to protect and promote healthy bone tissue in the mouth.

An exemplary lozenge composition can be prepared according to conventional means containing the following ingredients:

| | |
|---|---|
| Sorbitol Powder | 58.775% |
| Flavor and Color | 1.0 |
| Sweeteners | 0.2 |
| Tableting Lubricant | 5.0 |
| 1priflavone | 15.0 |
| Vanadyl Sulphate | 0.005 |
| Folic Acid | 0.020 |
| Calcium Citrate/Malate | 20.0 |

The ratio of ipriflavone to absobable calcium should be about 1 to 0.8, to 1:4. The ratio of ipriflavone to vanadium salt should be about 1 to 0.0001 to 1 to 0.030. The ration of ipriflavone to at least folic acid, if used as the homocysteine inhibiting agent, should be about 1 to 0.001 to 1 to 0.005, with these ratios applicable to the oral supplement, dentrifice, lozenge or mouthrinse to provide a general guideline for the various formulations.

While preferred embodiments of the present invention have been shown and described, it will be understood by those skilled in the art that various modifications can be made without varying from the spirit and scope of the invention. What is claimed is:

1. A composition for inhibiting bone loss and promoting bone rebuilding in a human or animal comprising a vanadium salt, ipriflavone, at least one homocysteine inhibiting agent and an absorbable calcium salt in a pharmaceutically acceptable carrier.

2. The composition of claim 1 wherein the composition comprises from 50-99% ipriflavone, from 0.01-0.2% vanadium salt, from 0.1-2.0% by weight homocysteine inhibitor and from 25-55% calcium salt.

3. The composition of claim 1 further comprising one or more optional ingredients selected from the group consisting of isoflavones such as from soy, red clover, or kudzu, pyridoxal-5phosphate, pyridoxine, various antioxidants, such as the oligomeric proanthocyanadins, vitamins, minerals, fiber, anti-inflammatory agents and nutritional supplements provided these do not detrimentally affect the bone building activity of the present invention or result in undesirable side effects.

4. The composition of claim 1 wherein the vanadium salt is vanadyl sulphate.

5. The composition of claim 1 wherein the homocysteine inhibiting agent is one or more of folic acid, vitamin B12, vitamin B6 and methyl donors selected from the group consisting of betaine (trimethylglycine), choline (tetramethylglycine), dimethylglycine, S-adenosyl-methionine (SAMe), S-adenosyl-cobalamine (dibencozide), and methyl sulfonyl methane (MSM).

6. The composition of claim 1 wherein the absorbable calcium salt is calcium citrate/malate/carbonate.

7. The composition of claim 1 comprising 50 mcg. of vanadium sulfate, 300 mg. of ipriflavone, 200 mcg. of folic acid, 100 mcg. of vitamin B 12, 15 mg. of pyridoxal-5-phosphate and 350 mg. of calcium citrate/malate/carbonate.

8. The compositions according to claim 1 wherein a unit dose of ipriflavone is between 300 and 600 mg and that the weight ratio of ipriflavone to carrier is at least 1:2.

9. A method for inhibiting bone loss and/or promoting bone rebuilding in a mammal comprising:
orally administering to the mammal an effective amount of a composition comprising a vanadium salt, ipriflavone, a homocysteine inhibiting agent and an absorbable calcium salt in a pharmaceutically acceptable carrier.

10. The method of claim 9 further comprising formulating the composition in a unit dose form.

11. The method of claim 9 wherein the composition is administered at from about 1 to 10 mg per kg of body weight.

12. The method of claim 9 wherein the composition is administered at from about 1 to 4 mg per kg of body weight.

13. The method of claim 9 wherein the composition is administered in multiple daily doses.

14. A method for preventing bone loss associated with aging and osteoporosis in a human or animal comprising orally administering to the human or animal a prophylactically effective amount of a composition comprising a vanadium salt, ipriflavone, a homocysteine inhibiting agent and an absorbable calcium salt in a pharmaceutically acceptable carrier.

15. The composition of claim 1, wherein the composition is formulated for administration in a form selected from the group consisting of dentrifice, a lozenge, chewing gum, a confection and a mouthrinse.

16. The composition of claim 1 wherein the composition is a dentrifice further comprising a pharmaceutically acceptable, topical, oral dentrifice carrier.
